(19) United States
(12) Patent Application Publication
(10) Pub. No.: US 2024/0216394 A1
(43) Pub. Date: Jul. 4, 2024

(54) GOLEXANOLONE FOR USE IN THE TREATMENT OF FATIGUE OR COGNITIVE IMPAIRMENT IN LIVER DISEASE PATIENTS

(71) Applicant: Umecrine Cognition AB, Solna (SE)

(72) Inventors: Magnus Doverskog, Stockholm (SE); Maja Johansson, Vannas (SE); Lars Öhman, Stockholm (SE); David Jones, Newcastle upon Tyne (GB)

(21) Appl. No.: 18/287,329

(22) PCT Filed: Apr. 19, 2022
(86) PCT No.: PCT/EP2022/060260

§ 371 Date: Oct. 18, 2023

(30) Foreign Application Priority Data
Apr. 20, 2021 (EP) 21169388.2

(51) Int. Cl. A61K 31/568; A61P 1/16

(52) U.S. Cl. CPC A61K 31/568; A61P 1/16

(57) ABSTRACT

The present invention is directed to the compound golexanolone or a pharmaceutically acceptable salt thereof for use in the treatment of fatigue or cognitive impairment, or a combination thereof, in a non-cirrhotic patient with a chronic liver disease, wherein the patient has a serum level of allopregnanolone which is 0.03 ng/ml or higher.

### FIELD OF THE INVENTION

The present invention is directed to the compound golexanolone, having the chemical name 3α-ethynyl-3β-hydroxyandrostan-17-one oxime, or a pharmaceutically acceptable salt thereof, for use in the treatment of fatigue or cognitive impairment, or a combination thereof, in a non-cirrhotic patient suffering from a chronic liver disease, wherein the patient has a serum level of allopregnanolone which is 0.03 ng/ml or higher.

### BACKGROUND OF THE INVENTION

Chronic liver diseases that inevitably lead to hepatic fibrosis, cirrhosis and/or hepatocellular carcinoma have become a major cause of illness and death worldwide. Among them, cholangiopathies or cholestatic liver diseases comprise a large group of conditions in which injury is primarily focused on the biliary system. These include congenital diseases (such as biliary atresia and cystic fibrosis), acquired diseases (such as primary sclerosing cholangitis and primary biliary cholangitis), and those that arise from secondary damage to the biliary tree from obstruction, cholangitis or ischaemia. These conditions are associated with a specific pattern of chronic liver injury centered on damaged bile ducts that drive the development of peribiliary fibrosis and, ultimately, biliary cirrhosis and liver failure. For most, there is no established medical therapy and, hence, these diseases remain one of the most important indications for liver transplantation. Liver cirrhosis accounts for a growing and substantial disease burden worldwide (G Rajapaksha et al; World J Gastrointest Pathophysiology 2019; January 5; 10(1): pp. 1-10).

Biliary diseases or cholangiopathies are a group of chronic liver diseases characterized by cholestasis and progressive biliary fibrosis that can lead to end stage liver failure. There are numerous aetiologies for these diseases. Two of the common cholangiopathies are immune disorders, primary biliary cholangitis (PBC) and primary sclerosing cholangitis (PSC). Many cholangiopathies, including PBC, primarily affect the bile ducts. Once bile flow is impaired, bile is accumulated in the liver, causing primary damage to the biliary epithelium and eventually the liver parenchyma. A majority of cholangiopathies have similar features, including peri-portal inflammations that lead to liver fibrosis/cirrhosis. Given their progressive nature, most cholangiopathies cause substantial morbidity and mortality in patients and they are a major indication for liver transplantation.

Primary Biliary Cholangitis (PBC), previously known as primary biliary cirrhosis, is an autoimmune chronic disease of the liver. It results from a slow, progressive destruction of the small and intermediate bile ducts of the liver, causing bile and other toxins to build up in the liver, a condition called cholestasis, leading to i.a. periportal inflammation, fibrosis and biliary cirrhosis. PBC predominantly affects women. Characteristic symptoms include pruritus, fatigue and an increasingly recognised mild cognitive impairment. Cognitive symptoms are however prevalent in PBC independent of liver disease severity, and it is believed that PBC-related cognitive impairment is unrelated to hepatic encephalopathy (J. L. Newton et al; Hepatology, Vo. 48, No.2 2008pp. 541-549).

Primary sclerosing cholangitis (PSC) is a long-term progressive disease of the liver and gallbladder characterized by inflammation and scarring of the bile ducts which normally allow bile to drain from the gallbladder. The bile duct scarring which occurs in PSC narrows the ducts of the biliary tree and impedes the flow of bile to the intestines. Eventually, it can lead to cirrhosis of the liver and liver failure. PSC increases the risk of various cancers including liver cancer, gallbladder carcinoma, colorectal cancer, and cholangiocarcinoma. The underlying cause of PSC is unknown and there is no approved therapy available.

Fatigue is one of the most common non-specific symptoms considered as a crucial issue in health care and has been associated with several chronic disease states including multiple sclerosis, systemic lupus erythematosus, rheumatoid arthritis, as well as chronic viral and cholestatic liver diseases. A particularly high prevalence of fatigue in patients with primary biliary cholangitiss (PBC; 86%) and chronic hepatitis C (CHC; 69%) has been reported. The cause of fatigue associated with liver disease is not well understood, and few studies have investigated this symptom (S. Ahboucha et al: Neurogastroenterol Motil (2008); 20, pp. 671-679). Aboucha report significant increase of circulating levels of the neurosteroid allopregnanolone in cirrhotic patients with primary biliary cholangitis (PBC) and chronic hepatitis C (CHC).

In addition, sleep abnormality, in the form of excessive daytime somnolence, is present in a significant proportion of patients with PBC, with the degree of daytime somnolence correlating strongly with the degree of fatigue (Newton J. L. et al., Hepatology, Vo. 44, No. 1 2006 pp. 91-98).

Fatigue is one of the most common symptoms of PBC. It occurs in up to 85% of patients (Huet P M, et al. Am J Gastroenterol. 2000; 95(3): pp.760-767), and is a particularly debilitating symptom affecting quality of life and resulting in social isolation. No medical treatment has been shown to be effective in alleviating this symptom. Currently there are no widely accepted or effective treatments for fatigue in patients suffering from a chronic liver disease such as Primary Biliary Cholangitis (PBC), Primary sclerosing cholangitis (PSC) or any other type of cholangiopathy. Drugs such as Modafinil, Fluoxetine and Rituximab have been tried in patients suffering from PBC-related fatigue, but neither have proven beneficial.

The compound golexanolone, having the chemical name 3α-ethynyl-3β-hydroxyandrostan-17-one oxime, is a compound currently in clinical Phase II for the treatment of Hepatic Encephalopathy (HE). This compound is disclosed in WO 2008/063128 for use in various CNS disorders. WO 2015/114308 disclose the use of the compound 3α-ethynyl-3β-hydroxyandrostan-17-one oxime for the treatment of Hepathic Encephalopathy (HE). US patent application published as US2017/0348323, discloses a method for the treatment of hypersomnolence by administering the compound 3α-ethynyl-3β-hydroxyandrostan-17-one oxime.

R. F. Butterworth et al report that the effect of DHEAS (dehydroepiandrosterone sulphate) had a positive effect on whole body fatigue in rats following bile duct ligation (BDL) (R. F. Butterworth et al; Neurogastroenterol Motil (2009) 21, pp. 1319-1325). DHEAS is a steroid hormone which is active on several receptors such as GABA_{A}-receptors, NMDA-receptors, sigma-1 receptors and metabotrophic glutamate receptors (mGluR), and also modulates a variety of neurotransmitter systems, including the cholinergic, GABAergic, dopaminergic and glutamatergic systems (Y. Dong et al: Journal of Neuroendocrinology 2011; 24, pp. 215-224).

There is a medical need for a drug which may be useful in therapy of fatigue in a patient suffering from a chronic liver disease such as a cholangiopathy. There is also a medical need for a drug which may be useful in therapy of cognitive impairment in a patient suffering from a chronic liver disease such as a cholangiopathy.

### DESCRIPTION OF THE INVENTION

A problem underlying the present invention, is to find a novel therapy that may be useful for the treatment of fatigue or cognitive impairment, or a combination thereof, in a non-cirrhotic patient suffering from a chronic liver disease.

One aspect of the present invention, is the compound golexanolone (3α-ethynyl-362 -hydroxyandrostan-17-one oxime) of formula (I)

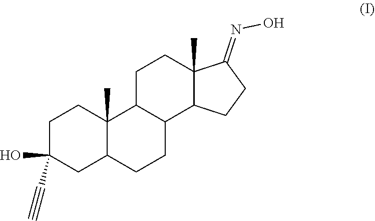 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of fatigue or cognitive impairment, or a combination thereof, in a non-cirrhotic patient with a chronic liver disease, said patient having a serum level of allopregnanolone which is 0.03 ng/ml or higher.

In yet an aspect of the invention, the compound golexanolone (3α-ethynyl-3β-hydroxyandrostan-17-one oxime) is for use in the treatment of fatigue or cognitive impairment or excessive daytime sleepiness (EDS), or any combination thereof, in a non-cirrhotic patient with a chronic liver disease which is a cholangiopathy such as primary biliary cholangitis (PBC) or primary schlerosing cholangitis (PSC); chronic hepatitis B (CHB); chronic hepatitis C (CHC); non-alcoholic fatty liver disease (NAFLD); or non-alcoholic steatohepatitis (NASH); and wherein the patient has a serum level of allopregnanolone which is 0.03 ng/ml or higher.

Yet an aspect of the invention, is the compound golexanolone (3α-ethynyl-3β-hydroxyandrostan-17-one oxime) for use in the treatment of fatigue or cognitive impairment, or a combination thereof, in a non-cirrhotic patient with a cholangiopathy, and wherein the patient has a serum level of allopregnanolone which is 0.03 ng/ml or higher.

Yet an aspect of the invention, is the compound golexanolone (3α-ethynyl-3β-hydroxyandrostan-17-one oxime) for use in the treatment of fatigue or cognitive impairment, or a combination thereof, in a non-cirrhotic patient with primary biliary cholangitis (PBC), and wherein the patient has a serum level of allopregnanolone which is 0.03 ng/ml or higher.

In one aspect of the invention, a non-cirrhotic patient with primary biliary cholangitis (PBC) as described and claimed herein, also suffers from itching.

In yet an aspect of the invention, a non-cirrhotic patient with primary biliary cholangitis (PBC) as described and claimed herein, also suffers from social problems.

In yet an aspect of the invention, a non-cirrhotic patient with primary biliary cholangitis (PBC) as described and claimed herein, also suffers from emotional problems.

In one aspect of the invention, the PBC patient as herein described and claimed has a PBC-40 Cognitive Domain which is 15 or higher(moderate).

In an aspect of the invention, the PBC patient as herein described and claimed has a PBC-40 Cognitive Domain which is in the range 15-21.

In yet an one aspect of the invention, the PBC patient as herein described and claimed has a PBC-40 Cognitive Domain which is 21 or higher (severe).

In one aspect of the invention, the PBC patient as herein described and claimed has a PBC-40 Fatigue Domain which is 28 or higher (moderate).

In yet an aspect of the invention, the PBC patient as herein described and claimed has a PBC-40 Fatigue domain which is in the range 28-39.

In yet an aspect of the invention, the PBC patient as herein described and claimed has a PBC-40 Fatigue domain which is 39 or higher (severe).

In yet an aspect of the invention, the PBC patient as herein described and claimed has a PBC-40 Itching Domain which is in the range 3-15.

In yet an aspect of the invention, the PBC patient as herein described and claimed has a PBC-40 Itching Domain which is 15 or higher.

Yet an aspect of the invention, is the compound golexanolone (3α-ethynyl-3β-hydroxyandrostan-17-one oxime) for use in the treatment of fatigue or cognitive impairment, or a combination thereof, in a non-cirrhotic patient with primary schlerosing cholangitis (PSC), and wherein the patient has a serum level of allopregnanolone which is 0.03 ng/ml or higher.

Yet an aspect of the invention, is the compound golexanolone (3α-ethynyl-3β-hydroxyandrostan-17-one oxime) for use in the treatment of fatigue or cognitive impairment, or a combination thereof, in a non-cirrhotic patient with chronic hepatitis C (CHC), and wherein the patient has a serum level of allopregnanolone which is 0.03 ng/ml or higher.

Yet an aspect of the invention, is the compound golexanolone (3α-ethynyl-3β-hydroxyandrostan-17-one oxime) for use in the treatment of fatigue or cognitive impairment, or a combination thereof, in a non-cirrhotic patient with chronic hepatitis B (CHB), and wherein the patient has a serum level of allopregnanolone which is 0.03 ng/ml or higher.

Yet an aspect of the invention, is the compound golexanolone (3α-ethynyl-3β-hydroxyandrostan-17-one oxime) for use in the treatment of fatigue or cognitive impairment, or a combination thereof, in a non-cirrhotic patient with nonalcoholic fatty liver disease (NAFLD), and wherein the patient has a serum level of allopregnanolone which is 0.03 ng/ml or higher.

Yet an aspect of the invention, is the compound golexanolone (3α-ethynyl-3β-hydroxyandrostan-17-one oxime) for use in the treatment of fatigue or cognitive impairment, or a combination thereof, in a non-cirrhotic patient with nonalcoholic steatohepatitis (NASH), and wherein the patient has a serum level of allopregnanolone which is 0.03 ng/ml or higher.

One aspect of the invention, is the compound golexanolone (3α-ethynyl-3β-hydroxyandrostan-17-one oxime) for use in the treatment of fatigue or cognitive impairment, or a combination thereof, in a non-cirrhotic patient with pre-existing cirrhosis, and wherein the patient has a serum level of allopregnanolone which is 0.03 ng/ml or higher.

One aspect of the invention, is a method for the treatment of fatigue or cognitive impairment, or a combination thereof, in a non-cirrhotic patient with a chronic liver disease, wherein the patient has a serum level of allopregnanolone which is 0.03 ng/ml or higher, whereby the compound golexanolone (3α-ethynyl-3β-hydroxyandrostan-17-one oxime) of formula (I)

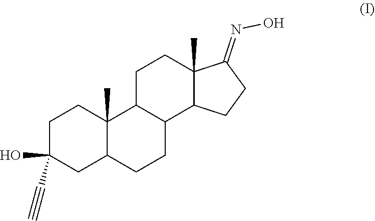 [SMILES: C#C[C@]1(O)CC[C@@]2(C)C(CCC3C2CC[C@]2(C)/C(=N/O)CCC32)C1]

or a pharmaceutically acceptable salt thereof, is administered to a patient suffering from said chronic liver disease.

Yet an aspect of the invention, is the compound golexanolone (3α-ethynyl-3β-hydroxyandrostan-17-one oxime) for use in the treatment of a symptom related to cholangiopathy, such as primary biliary cholangitis (PBC) or primary sclerosing cholangitis (PSC).

Yet an aspect of the invention, is the compound golexanolone (3α-ethynyl-3β-hydroxyandrostan-17-one oxime) for use in the treatment of a symptom related to a chronic liver disease selected from chronic hepatitis B (CHB), chronic hepatitis C (CHC), nonalcoholic fatty liver disease (NAFLD), nonalcoholic steatohepatitis (NASH), and pre-existing cirrhosis.

Yet an aspect of the invention is a therapy against a symptom related to a chronic liver disease as herein disclosed and claimed, and which may provide a therapeutic effect.

Yet an aspect of the invention is the use of the compound golexanolone (3α-ethynyl-3β-hydroxyandrostan-17-one oxime) of formula (I)

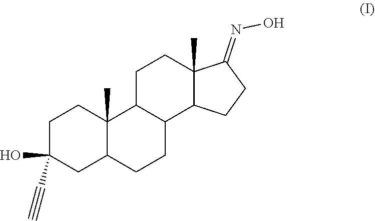 [SMILES: C#C[C@]1(O)CC[C@@]2(C)C(CCC3C2CC[C@]2(C)/C(=N/O)CCC32)C1]

or a pharmaceutically acceptable salt thereof, for the manufacture of a medicament for use in the treatment of fatigue or cognitive impairment, or a combination thereof, in a non-cirrhotic patient with a chronic liver disease, wherein the patient has a serum level of allopregnanolone which is 0.03 ng/ml or higher.

In one aspect of the invention, the compound golexanolone (3α-ethynyl-3β-hydroxyandrostan-17-one oxime) is used as monotherapy.

In yet an aspect of the invention, the compound golexanolone (3α-ethynyl-3β-hydroxyandrostan-17-one oxime) may be used in combination with at least one more compound useful for the treatment of fatigue or cognitive impairment, or a combination thereof, in a non-cirrhotic patient suffering from a chronic liver disease and wherein the patient has a serum level of allopregnanolone which is 0.03 ng/ml or higher, such as a chronic liver disease selected from cholangiopathy such as primary biliary cholangitis (PBC) or primary sclerosing cholangitis (PSC), chronic hepatitis B (CHB), chronic hepatitis C (CHC), nonalcoholic fatty liver disease (NAFLD), nonalcoholic steatohepatitis (NASH), and pre-existing cirrhosis.

In one aspect of the invention, combination therapy may be as add-on therapy. In yet an aspect of the invention, the compound golexanolone (3α-ethynyl-3β-hydroxyandrostan-17-one oxime) may be administered as a fix combination (such as a pharmaceutical formulation) or by administration sequentially (i.e. after one another).

Medications which may be useful in combination therapy with the compound golexanolone (3α-ethynyl-3β-hydroxyandrostan-17-one oxime), is a medication or several medications, or a compound or several compounds, used as standard of care treatment of a patient with a chronic liver disease such as cholangiopathy, such as primary biliary cholangitis (PBC) or primary sclerosing cholangitis (PSC), chronic hepatitis B (CHB), chronic hepatitis C (CHC), nonalcoholic fatty liver disease (NAFLD), nonalcoholic steatohepatitis (NASH), pre-existing cirrhosis or cirrhosis.

Examples of medications or compounds which may be useful in combination therapy with the compound golexanolone (3α-ethynyl-3β-hydroxyandrostan-17-one oxime) of PBC-related fatigue or cognitive impairment, or a combination thereof, in a non-cirrhotic patient and wherein the patient has a serum level of allopregnanolone which is 0.03 ng/ml or higher, are oral antioxidants, fluvoxamine, fluoxetine, ondansetron, colchicine, UDCA (ursodeoxycholic acid), OCA (obeticholic acid), ciclosporin, nalmefene, modafinil, rituximab, lactulose, rifaximin, propranolol, furosemide, a fibrates (such as bezafibrate and fenofibrate), methotrexate, or any combination thereof. Budesonide is yet an example of a compound which may be used in combination therapy with the compound golexanolone as herein described and claimed.

The compound golexanolone (3α-ethynyl-3β-hydroxyandrostan-17-one oxime) may be prepared according to the method of preparation disclosed in WO 2008/063128.

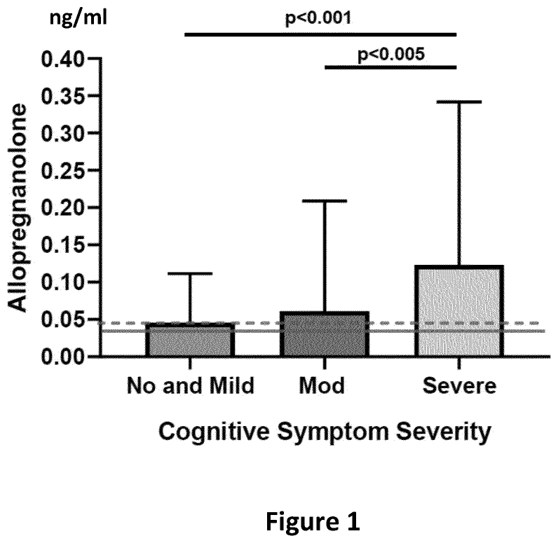

FIG. 1 illustrates the correlation between the severity of cognitive symptoms and the level of the neurosteroid allopregnanolone in serum of PBC patients.

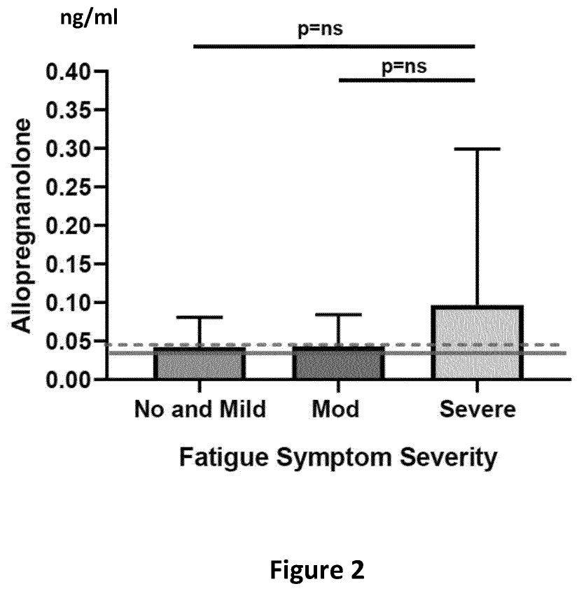

FIG. 2 illustrates the correlation between the severity of fatigue symptoms and the level of the neurosteroid allopregnanolone in serum of PBC patients.

### DEFINITIONS

Cholangipathy (or cholangiopathies) is defined as a group of diseases that mainly target the biliary tree and which includes a group of chronic liver diseases characterized by cholestasis and progressive biliary fibrosis that can lead to end stage liver failure. Cholangipathy (or cholangopathies) can be divided into several categories according to the pathogenetic mechanism involved. However, in many cholangiopathies, more than one pathogenetic mechanism is operative:
(1) Immune-mediated cholangiopathy
(2) Infectious cholangiopathy
(3) Genetic cholangiopathy
(4) Ischemic cholangiopathy
(5) Drug- or toxin-induced cholangiopathy

Two of the most common cholangiopathies are primary biliary cholangitis (PBC) and primary sclerosing cholangitis (PSC) respectively. PBC belongs to the group of immune mediated cholangiopathies.

Primary biliary cholangitis is a chronic, seropositive and female-predominant inflammatory and cholestatic liver disease, which has a variable rate of progression towards biliary cirrhosis. Primary Biliary Cholangitis (PBC) is often considered a model of autoimmune disease because of its hallmark serologic signature, the antimitochondrial antibody and specific bile duct pathology. In addition to the risks of progression to cirrhosis and end-stage liver disease, PBC is characterised by significant symptoms of cognitive dysfunction and linked central fatigue which have a substantial impact on patient quality of life.

PBC patients experience a significant additional problem set with the development of central fatigue or cognitive dysfunction, or a combination of central fatigue and cognitive dysfunction, and related symptoms to these, in addition to the liver disease progression itself. The impact on quality of life is often significant, and is disproportionately high in younger patients adding to the level of disability. In contrast to disease progression, no progress has been made in treating this problem and this is a major area of unmet medical clinical need.

Cognitive symptoms, fatigue, excessive daytime sleepiness, or any combination thereof, are present throughout cholangiopathy disease course in affected patients and are unrelated to conventional markers of disease severity, suggesting that they are unrelated to hepatic encephalopathy, and to advanced cirrhotic disease. There is also evidence of organic brain change in terms of impaired performance on formal cognitive testing, anatomical and functional change on MR, and neurophysiological assessment using transcranial magnetic stimulation.

Excessive Daytime Sleepiness (EDS) is defined as the inability to stay awake and alert during major waking episodes of the day, resulting in periods of irrepressible need for sleep or unintended lapses inta drowsiness or sleep. The Epworth Sleepiness Scale (ESS) is widely used in the field of sleep medicine as a subjective measure of a patient's sleepiness (Johns 1991; Sleep, 14(6), pp. 540-545). The total ESS score can range from zero to 24 with higher scores correlating with increasing degrees of sleepiness.

Primary sclerosing cholangitis (PSC) is a long-term progressive disease of the liver and gallbladder characterized by inflammation and scarring of the bile ducts which normally allow bile to drain from the gallbladder. The bile duct scarring which occurs in PSC narrows the ducts of the biliary tree and impedes the flow of bile to the intestines. Eventually, it can lead to cirrhosis of the liver and liver failure.

Chronic hepatitis is an inflammation of the liver that lasts for at least 6 months. Many people have no symptoms, but others have vague symptoms, such as a general feeling of illness, poor appetite, and fatigue. Chronic hepatitis can result in cirrhosis with portal hypertension and liver failure. Both chronic hepatitis B (CHB) and chronic hepatitis C (CHC) are caused by a virus infection characterised by varying degrees of inflammation and hepatic fibrosis.

Nonalcoholic fatty liver disease (NAFLD) is an umbrella term for a range of liver conditions affecting people who drink little to no alcohol. Excess fat builds up in the liver due to causes other than alcohol use. NAFLD is marked by liver inflammation, which may progress to scarring and irreversible damage. This damage is similar to the damage caused by heavy alcohol use. Nonalcoholic steatohepatitis (NASH) is the most severe form of non-alcoholic fatty liver disease (NAFLD), and is characterized by the presence of an abnormal accumulation of fat in the liver which in some individuals can progress to liver cell injury (hepatocellular ballooning) and inflammation. As NASH evolves, over time it can result in excessive scarring in the liver (fibrosis), a natural response to injury which can lead to liver cirrhosis or liver cancer.

Cirrhosis is a progressive disease, developing slowly over many years. If it is allowed to continue, the buildup of scar tissue can eventually stop liver function. For cirrhosis to develop, long-term, continuous damage to the liver needs to occur. When healthy liver tissue is destroyed and replaced by scar tissue, the condition becomes serious, because it can start blocking the flow of blood through the liver.

Patients suffering from a chronic liver disease, often also suffers from symptoms such as fatigue, cognitive impairment or a combination of fatigue and cognitive impairment.

Fatigue in a patient suffering from a chronic liver disease including cholangiopathy such as primary biliary cholangitis (PBC) or primary sclerosing cholangitis (PSC), chronic hepatitis B (CHB), chronic hepatitis C (CHC), nonalcoholic fatty liver disease (NAFLD), nonalcoholic steatohepatitis (NASH), or pre-existing cirrhosis, are each within the scope of the present invention.

Patients suffering from a chronic liver disease such as PBC or PSC and who suffers from symptoms such as fatigue, or cognitive impairment (brain fog), or a combination thereof, has a pathophysiology which is different from a patient suffering from Hepathic Encephalopathy.

Characteristic symptoms of PBC include pruritus, fatigue and/or an increasingly recognised mild cognitive impairment. Cognitive symptoms are however prevalent in PBC independent of liver disease severity, and it is believed that PBC-related cognitive impairment is unrelated to hepatic encephalopathy (J. L. Newton et al; Hepatology, Vo. 48, No.2 2008; pp. 541-549).

Fatigue in patients suffering from PBC, have both peripheral and central fatigue components. The central component (“brain fog”) is characterised by neurophysiological abnormalities and is related to cognitive symptoms and sleep pattern disturbance. The peripheral component means an inability to sustain physical activity, loss of energy or subjects feeling that they are “running out of fuel”.

Cognitive impairment (reduced cognitive performance), whilst also being associated with fatigue, is a significant discrete symptom in patients suffering from PBC.

The disease terminology and symptom terminology as used throughout the specification and claims, is terminology in the field of chronic liver diseases applicable at the priority date of the present patent application. Terminology in the medical filed of liver diseases may evolve in the future for diseases such as PBC, NASH, schlerosing cholangitis (PSC), NAFLD, and NASH, but this should not change the scope of protection as herein described and claimed since the diseases and symptoms will still be the same.

The wording “treatment or therapy” as used throughout the specification and claims, takes the normal wording within the medical and pharmaceutical field, and means treatment of fatigue or cognitive impairment, or a combination thereof, in a non-cirrhotic patient who has been diagnosed as having (suffering from) a chronic liver disease as disclosed and claimed herein.

The wording “monotherapy” as used herein, means therapy with the compound golexanolone (3α-ethynyl-3β-hydroxyandrostan-17-one oxime) alone, administered to a non-cirrhotic patient who is diagnosed or has been diagnosed with a chronic liver disease as disclosed and claimed herein.

The wording “combination therapy” as used herein, means treatment (therapy) with the compound golexanolone (3α-ethynyl-3β-hydroxyandrostan-17-one oxime) in combination with an agent (drug) used as standard of care therapy.

Combination therapy with an agent (drug) used as standard of care therapy according to the invention, means that the compound golexanolone (3α-ethynyl-3β-hydroxyandrostan-17-one oxime) may be administered prior to, after (add-on) or simultaneously to the administration of an agent (drug) used as standard of care therapy for a chronic liver disease as herein disclosed and claimed.

Add-on therapy as used herein, is defined as combination therapy wherein the compound golexanolone (3α-ethynyl-3β-hydroxyandrostan-17-one oxime) is administered to a subject who is already being treated with a medication indicated for fatigue in a patient with a chronic liver disease such as cholangiopathy, such as primary biliary cholangitis (PBC) or primary sclerosing cholangitis (PSC), chronic hepatitis B (CHB), chronic hepatitis C (CHC), nonalcoholic fatty liver disease (NAFLD), nonalcoholic steatohepatitis (NASH), or pre-existing cirrhosis.

A fix combination as used herein, may in one aspect be defined as a combination where the compound golexanolone (3α-ethynyl-3β-hydroxyandrostan-17-one oxime) is formulated in admixture with at least one more compound useful in the treatment of fatigue in a patient suffering from a chronic liver disease selected from cholangiopathy such as primary biliary cholangitis (PBC) or primary schlerosing cholangitis (PSC), chronic hepatitis B (CHB), chronic hepatitis C (CHC), nonalcoholic fatty liver disease (NAFLD), nonalcoholic steatohepatitis (NASH), and pre-existing cirrhosis. In yet an aspect, a fix combination may be defined as a “kit of parts” combination where each active ingredient or formulation may be administered simultaneously (i.e. in parallell but as separate formulations) or one after the other (sequentially).

The wording “treatment of fatigue or cognitive impairment, or a combination thereof” as used throughout the present specification and claims, means that a patient suffering from a chronic liver disease as disclosed and claimed herein, may suffer from only the symptom fatigue or only the symptom cognitive impairment, or from both the symptoms fatigue and cognitive impairment.

As used throughout the present specification and claims, the compound golexanolone is the compound with the chemical name 3α-ethynyl-3β-hydroxyandrostan-17-one oxime, according to the chemical formula (I),

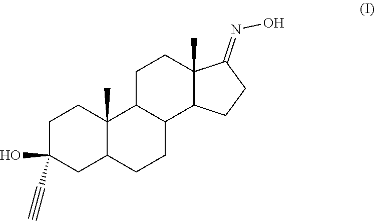 [SMILES: C#C[C@]1(O)CC[C@@]2(C)C(CCC3C2CC[C@]2(C)/C(=N/O)CCC32)C1]

or a pharmaceutically acceptable salt thereof.

The wording “golexanolone” is the International Nonproprietary Name (INN) for the compound 3α-ethynyl-3β-hydroxyandrostan-17-one oxime, and this compound has the CAS no. 2089238-18-4.

The wording “serum” as used herein, means blood plasma without fibrinogens.

The wording “PBC-40” as used in accordance with the present invention, means a patient-derived, disease specific quality of life measure developed and validated for use in PBC (Jacoby A, Rannard A, Buck D, Bhala N, Newton J L, James O F W, Jones D E J. Development, validation and evaluation of the PBC-40, a disease specific health related quality of life measure for primary biliary cirrhosis. Gut 2005;54: pp. 1622-1629). There are six domains of PBC-40 which relate to fatigue, emotional, social, cognitive function, general symptoms, and itch. The measure has been fully validated for use in PBC and shown to be scientifically sound. The wording “PBC-40 Fatigue Domain”, “PBC-40 Cognitive Domain”, PBC-40 Itching Domain”, “PBC-40 Social Domain”, and “PBC-40 Emotional Domain” respectively and as used in accordance with the present invention, are defined as disclosed in Jacoby et al (Gut 2005;54: pp. 1622-1629).

The wording “brain fog” as used herein, means difficulty in processing information, acting on it and remembering it.

### PHARMACEUTICAL FORMULATIONS AND ADMINISTRATION ROUTES

In certain aspects of the invention, the compound golexanolone (3α-ethynyl-3β-hydroxyandrostan-17-one oxime) as used throughout the present specification and claims, may be administered in the form of a pharmaceutical composition, in admixture with one or more pharmaceutically acceptable adjuvants, diluents and/or carriers. Examples of such pharmaceutically acceptable excipients, carriers and/or diluents useful when formulating the compound golexanolone (3α-ethynyl-3β-hydroxyandrostan-17-one oxime) for use in accordance with the present invention, are thickeners, flavoring agents, diluents, emulsifiers, dispersing aids, carrier substances, lubricants or binders. Typical pharmaceutical carriers include, but are not limited to, binding agents (e.g., pregelatinised maize starch); fillers (e.g., lactose, glucose, sucrose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, and sodium starch glycolate); wetting agents; diluents; coloring agents; emulsifying agents; pH buffering agents; preservatives; and mixtures thereof.

In one aspect of the invention, the compound golexanolone (3α-ethynyl-3β-hydroxyandrostan-17-one oxime) may be administered by enteral administration when used as disclosed and claimed herein. Examples of enteral administration involves administration to the esophagus, stomach, and small and large intestines (i.e., the gastrointestinal tract). Methods of administration include oral, sublingual (dissolving the drug under the tongue), and rectal.

The physician will be able to determine the actual dosage of the compound golexanolone (3α-ethynyl-3β-hydroxyandrostan-17-one oxime) which will be suitable for an individual patient in order to treat a chronic liver disease symptom selected from fatigue, cognitive impairment or a combination of both these symptoms, which dosage may vary with the route of administration, the type and severity of the symptom and patient population to be treated, as well as the species, age, weight, sex, and severity of the disease.

In one aspect of the invention, the compound golexanolone (3α-ethynyl-3β-hydroxyandrostan-17-one oxime) may be administered as a daily dose of from 1 mg to 200 mg, 10 mg to 100 mg, 3 mg to 30 mg, 30 mg to 60 mg, 50 mg to 100 mg, 20 mg to 160 mg, 40 mg to 160 mg, or 80 mg to 160 mg.

The wording “daily dose” may be administration of the compound golexanolone (3α-ethynyl-3β-hydroxyandrostan-17-one oxime) once daily, or twice daily (B.I.D.). Administration twice daily (B.I.D.) means that the total daily dose is divided into two doses which in total makes up the daily dose. For example, a daily dose of 1 mg to 200 mg may be administered as a dose of 1-200 mg once daily, or as a dose of 0.5-100 mg twice daily (B.I.D.).

In one aspect of the invention, a daily dose of the compound golexanolone (3α-ethynyl-3β-hydroxyandrostan-17-one oxime) which may be useful in accordance with the present invention may be selected from any one of 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, and 160 mg.

### BIOLOGICAL EVALUATION

Inventors of the present invention have found and demonstrated that non-cirrhotic PBC patients with severe cognitive symptoms, have a significant elevation of the neurosteroid compound allopregnanolone in serum, and more particularly a serum level of allopreganolone which exceeds 0.03 ng/ml.

### Background and Aims

Rice S et al. report findings which indicate that fatigue, bone ache, and memory and concentration problems have the greatest impact on patient health-related quality of life (HRQoL) but being associated with low health service costs. In contrast, PBC complications have little additional effect on HRQoL while being associated with significant health service costs, except for ascites. These significant HRQoL and cost effects, accounting for current treatment, suggest the potential for benefit from new treatments (Rice S et al; Clin Gastroenterol Hepatol 2021; 19: pp. 768-776).

Allopregnanolone (AP), a neurosteroid, is a potent positive allosteric modulator of GABAA receptors. It is linked to mood disorders with adverse effects on cognition and memory. This study explored associations between allopregnanolone (AP) and a disease-specific quality of life scoring system (PBC-40) in PBC patients.

### EXAMPLE 1

### Method

Serum AP levels were measured using mass spectrometry (detection limit 0.002ng/ml, quantitation limit 0.005 ng/ml) in 160 subjects: 120 fully-phenotyped PBC patients from the UK-PBC (30) and BANC (90) studies and 40 age- and gender-matched healthy controls. All PBC patients completed the PBC-40 at recruitment. Serum AP was compared across the PBC-40 domains for those with no or mild symptoms (comparable to symptoms found in the general population) versus severe symptoms.

### Results

There were no significant differences in AP levels between healthy controls (median=0.03 ng/ml [IQR=0.025]) and PBC (0.031 [0.42]), p=0.42, or between males (0.025 [0.033]) and females (0.032 [0.037]), p=0.57. Younger age was predictive of significantly higher AP levels within the PBC cohort (X2(2)=27.7, p<0.001) but not in healthy controls (X2(2)=6.0, p=0.119). There were significant differences within three PBC-40 domains: cognition (u=1034, p=0.02), emotional (u=1374, p=0.004) and itch (u=795, p=0.03). Younger age was associated with severe cognitive symptoms: severe (50 [12]) vs. none (60 [13]; p=0.001).

### Conclusion

As shown in FIG. 1, PBC patients having a higher serum level of allopregnanolone, have more severe cognitive symptoms. The solid horizontal line is the mean for the normals in the cohort, the dotted horizontal line the upper 95% CI for the normal. The group “No and mild symptom” in FIG. 1 are normal, which fits with all PBC-40 domains where “mild” symptoms are equivalent to ambient symptoms in the normal population. Severe are markedly affected and moderate are in the middle. Severe cognitive symptoms are only seen in a minority (10-15%) but are devastating, especially in younger patients. These are the people struggling to keep their jobs. The cut-offs for moderate and severe cognitive symptoms predict well the degree of Quality of Life (QoL) impairment (Jacoby et al; Gut 2005;54: pp. 1622-1629).

As shown in FIG. 2, PBC patients having a higher serum level of allopregnanolone, have more severe Fatigue symptoms. The group “No and mild symptom” in FIG. 2 are normal, which fits will all PBC-40 domains where “mild” symptoms are equivalent to ambient symptoms in the normal population. Severe are markedly affected and moderate are in the middle. Severe Fatigue symptoms are only seen in a minority (10-15%) but are devastating, especially in younger patients.

### EXAMPLE 2

### Assessing the Eeffects of the Compound Golexanolone (3α-ethynyl-3β-hydroxyandrostan-17-one Oxime) on Fatigue and Cognitive Function in a Bile Duct Ligation (BDL) Model of Cholestatis

The BDL model is a validated model (Frissen et al. Hepatology 4 Aug 2020; https://doi.org/10.1002/hep.31494) which has previously been used to model fatigue in mice and has also been shown by Newcastle researchers to induce cognitive decline.

Cholestatic liver disease with moderate fibrosis is induced in mice using the bile duct ligation (BDL) model, where the bile duct is surgically ligated to restrict bile flow. Animals develop biliary obstruction that promotes inflammation and cholestasis followed by hepatocyte damage and then bile duct proliferation.

Assessment of the impact of the compound golexanolone (3α-ethynyl-3β-hydroxyandrostan-17-one oxime) on activity and cognitive function in the context of cholestatic liver disease is made.

### Animals

Adult C57BI6 male mice of 10 weeks of age with average weight of around 25 grams (Harlan UK), are used. The animals are maintained with 12:12h light-dark cycles with free access to food and water.

### BDL Surgical Procedure

Bile duct ligation (BDL) and sham surgery is performed under sterile conditions in a surgical procedure room. Animals are anaesthetized with isoflurane and buprenorphine is administered subcutaneously as an analgesic. In the sham surgical procedure, a laparotomy is performed followed by abdominal muscle and skin closure. The BDL procedure is as described for the sham surgery but laparotomy is followed by exposure and resection of the common bile duct, thereafter abdominal and skin wound closure. Immediately following surgery, all animals are housed in a heated laminar flow ventilated cabinet maintained at 25° C. where they are carefully observed.

### Application and Dosage of the Compounds

Vehicle or the compound golexanolone (3α-ethynyl-3β-hydroxyandrostan-17-one oxime), is incorporated into diet for the duration of the study.

### Study Groups

Three (3) different groups with n=6/12 mice (total n=30) are included in the BDL model as shown below.

| | | Treat- | | | Follow | Number |
|---|---|---|---|---|---|---|
| | Sur- | ment | Treat- | Commence | up/ | of |
| Group | gery | types | ment | treatment | harvest | animals |
| 1 | Sham | Vehicle | In diet | Day 0 | Day 10 | 6 |
| 2 | BDL | Vehicle | In diet | Day 0 | Day 10 | 12 |
| 3 | BDL | golexanolone | In diet | Day 0 | Day 10 | 12 |

Mice are kept in a warm cabinet post surgery and monitored twice daily for physical condition. Bodyweight is measured at baseline and daily in order to ensure that no significant weight loss takes place due to surgery.

### Standardised Behavioural Testing

Mice are characterised using two standardised behavioural tests for the assessment of both short-term and long-term memory, as well as activity and exploratory behaviour on post-surgical days 4, 9 and 10.

The open field test (MouseTrapp, Neurolytical, USA) is performed at day 4 and 9. The open field test uses touchscreen technology to automatically record and score small animal movement and behaviour over a 5-minute duration.

Y-maze testing at day 10 is used to characterize cognitive dysfunction. The animal's preference for the ‘novel’ arm versus the familiar arms (proportional time in novel arm) is used as a measure of spatial memory.

Ethovision video tracking software is used to track movement of the mice in the open field test (anxiety/activity test; primary activity outputs—number of steps, distance travelled) and γ-maze (short term/spatial memory test; percentage of time in novel arm).

### Organ Harvest

Brain tissue is collected (processed for histology and snap frozen) for future studies as may be required. Typical histological outputs may include—Immuno-fluorescence staining of neural populations e.g. neurons, interneurons, Sox2 positive progenitor, astrocytes or microglial cells.

Blood is taken for future studies as may be required. Typical outputs are AST (liver damage marker) and ALP (Biliary damage marker).

Bile is collected from the enlarged gall bladder and snap frozen for future studies as may be required (e.g. bile acid composition etc). Liver tissue is collected (processed for histology and snap frozen) for future studies as may be required. Typical histology outputs may include staining with Picrosirius Red and a-SMA to assess fibrosis and myofibroblast activation or keratin 19 (K19) for ductular proliferation.

Blood samples are drawn from the animals for analysis of the levels of allopregnanolone.

### EXAMPLE 3

### Bile Duct Ligation (BDL) Model of Cholestasis in Rats

Since the early '90s a main goal of the hepatology research has been the development of different animal models to unveil novel pathogenetic concepts and, consequently, to test in vivo efficacy of new treatment strategies. Among them, bile duct ligation (BDL) has been the first and most extensively used experimental model of cholestasis. First developed in 1932 by Cameron and Oakley, BDL is a surgically created animal model, performed in rodents, consisting of ligation/excision of the common bile duct. BDL was initially described in rats, but since then it has been more suitably adapted to mice. BDL has many advantages, such as the simple technical procedure, which can be easily reproduced, the low cost, and the rapid liver damage development, which makes experimental protocols shorter and thus, more convenient. In BDL, liver phenotype is characterized by well-established features of cholestatic damage, such as cholangiocyte proliferation resulting in exuberant ductular reaction, portal inflammation and brisk establishment of biliary fibrosis (Mariotti V. et al; BBA—Molecular Basis of Disease 1865 (2019) 954-964).

The most frequently used surgery-induced animal model of cholestasis relies on bile duct ligation (BDL), where a ligature is placed or surgical ligation is performed on the common bile duct. This surgical procedure creates an extrahepatic obstruction of the biliary system, which results in cholestasis and inflammation (Gijbels E. et al; Liver International. 2021;41: pp. 656-682).

### Effects of Golexanolone on Fatigue, Itching, Cognitive and Motor Function, Liver Damage, Peripheral Inflammation and Allopregnanolone Levels in a Bile Duct Ligation (BDL) Model of Cholestasis in Rats

Cholestasis is induced in rats using the bile duct ligation (BDL) surgery, where the bile duct is ligated to restrict bile flow. Animals develop biliary obstruction that promotes inflammation and cholestasis followed by hepatocyte damage.

The effects of golexanolone is assessed on fatigue, scratching (itching) and cognitive and motor function in BDL rats.

The effects on peripheral inflammation are evaluated by analysing key interleukins.

The effects on golenaxolone on liver damage are also evaluated, by analysing:
Peripheral markers in blood: Alkaline phosphatase, transaminases, bilirubin, bile acids
Liver damage by histological analysis: fibrosis, inflammation
Allopregnanolone levels in blood

Rats are sacrificed at 28 days from start of the study.

Two sets of experiments are performed, including 6 rats per group (24 rats) in each set.

### Animals

Male Wistar rats (100-150 g) (Charles River), are used. All rats are non-cirrhotic when entering the study. The animals are maintained with 12:12h light-dark cycles with free access to food and water.

### BDL Surgical Procedure

Bile duct ligation (BDL) and sham surgery is performed under sterile conditions in a surgical procedure room. Animals are anaesthetized with isoflurane and buprenorphine is administered subcutaneously as an analgesic. For BDL, laparotomy is followed by exposure, doubly ligated and resected between the ligatures, of the common bile duct. Thereafter, abdominal and skin wound closure is performed. Immediately following surgery, all animals are housed in a heated laminar flow ventilated cabinet maintained at 25° C. where they are carefully observed. In the sham surgical procedure, a laparotomy is performed and bile duct identification and manipulation without ligation or resection, followed by abdominal muscle and skin closure.

Five days after surgery, rats are cholestatic. Four weeks after surgery rats are cirrhotic.

The rats are killed around 30 days after surgery and plasma.

The effects on liver function is confirmed by pathological and biochemical analysis.

### Application and Dosage of the Compounds

Vehicle or the compound golexanolone (3α-ethynyl-3β-hydroxyandrostan-17-one oxime) is administrated once daily through intra-gastric probes. The treatment is started at day 6 after surgery and maintained for the duration of the study.

Golexanolone is administered to the rats as a pharmaceutical formulation which comprises golexanolone formulated in a mixture of mono -and diglycerides of capric/caprylic acid (Capmul® MCM EP/NF; Barentz ApS Odense, DK).
(i) Golexanolone is administered in an amount of 50 mg/kg as a solution of 1.25 mL/kg (40 mg/ml of golexanolone in a mixture of mono -and diglycerides of capric/caprylic acid as described above).
(ii) The vehicle (placebo) is a mixture of mono -and diglycerides of capric/caprylic acid as described above without golexanolone, which is administered with a volume of 1.25 mL/kg.

### Study Groups

Four (4) different groups with n=12 rats (total n=48) are included in the study as shown below. As mentioned above, the study is performed in two consecutive experiments using 24 rats in each experiment.

| | | | Number of |
|---|---|---|---|
| Group | Surgery | Treatment | animals |
| 1 | Sham | Vehicle | 12 |
| 2 | Sham | golexanolone | 12 |
| 3 | BDL | Vehicle | 12 |
| 4 | BDL | golexanolone | 12 |

### Behavioural Testing

Rats are characterised using different standardised behavioural tests for the assessment of both spatial and non-spatial recognition memory:
Novel object location test (NOL); novel object recognition test (NOR); short-term memory in the Y maze; itching behaviour (scratching); fatigue as well as motor activity (OPEN FIELD) and coordination (Motorater/Catwalk); and anxiety (OPEN FIELD) behaviour, as described below (the day of the test may be slightly modified).
1. Itching evaluation is performed at days 7 and 22 post-surgery.
2. Fatigue is assessed at days 8 and 23 post-surgery.
3. Motor coordination is assessed on days 8-11 post-surgery.
4. Motor activity and anxiety is evaluated with the open field test at days 15 to 17 post-surgery.
5. Cognitive assessment is performed with NOL and NOR tests at days 17 and 18 post-surgery.
6. Short-term spatial memory test is performed in the Y maze at day 21 post-surgery.
7. Sacrifice and tissue obtention is performed around day 30 post-surgery.

1. Itching is evaluated by videotaping of scratching behaviour for 30-60 min. Spontaneous scratching will be quantified by counting the number of scratches (bouts) of any region of the body performed by fore- or hind paws.

2. Fatigue is measured with a treadmill consisting of a motorized conveyor belt divided into two parallel sectors, each with an electrified grid. To avoid foot shocks (0.5 mV, 1 mA, 0.3 Hz), the animals have to walk forward without falling off the belt.

At room temperature, rats are pre-trained for one day, first exploring for 3 min without starting. Then at 10 cm/s for 5 min and then at 20 cm/s for 5 min. The test is carried out on the next day.

The rats are placed on the stationary belt inclined at 5° with a gradually increasing speed of up to 30 cm/s for 5 min and continue at that speed for another 15 min (20 min total). A sensor measures the time spent on the belt. The value recorded during the last 15 minutes of each test (and the times it falls on the grid) is recorded. Butterworth et al, Neurogastroenterology and Motility, 2009; Dec; 21(12): pp.1319-1325. doi: 10.1111/j.1365-2982.2009.01356.x.

3. Motor coordination is assessed with the Motorater and CatWalk systems.

Motorater: A kinematic analysis of locomotor performance is conducted using MotoRater apparatus (TSE Systems, Germany). The rats are trained to cross an illuminated glass-walled corridor to reach the dark escape box at the end. 24 hour later rats are tested to cross a ladder. The performance is recorded from the bottom by a mobile high-speed camera at 200 frames per second (Zörner et al Nat. Methods, 2010; 7(9): pp. 701-708).

The CatWalk™ system measures various aspects of locomotor pattern. Based on the position, pressure, and surface area of each footfall, multiple parameters are calculated. Trials in which the animal stopped or changed direction are excluded from subsequent analysis. Three uninterrupted trials are performed. Paw print designations are assigned and data analysed using the CatWalk analysis software (v 7.1) (Lucas et al, Front. Cell Neurosci., 2015. 8: pp. 441).

4. Open field test is used to evaluate activity and anxiety. Automatically record and score rats movement and behaviour over a 5-minute duration is performed in an open-field arena (70×70×40 cm) of black painted wood.

5. Cognitive function is evaluated with Novel object recognition (NOR) and novel object location (NOL) memory tests. These tests are performed in an open-field arena (70×70×40 cm) of black painted wood with visuospatial cues on the walls as in Taoro-González et al, FASEB J, 2019; 33(9): pp. 9913-9928. The Rats are habituated during 5 days by allowing them freely explore the empty field arena for 5 min each day in order to avoid anxious behaviour in the subsequent tests. The NOL test is performed on day 6.

The NOR test is performed on day 7. In the sample phase of NOR, 2 identical objects are placed in the arena, and after freely exploring for 3 min, the rat is put into its cage for 6 h. After that, the test phase is performed, exchanging one of the objects for an unexplored object and allowing the rat to freely explore again for 3 min.

6. Short-term spatial memory test is performed using a black methacrylate Y-maze. The rat is placed into one of the arms of the maze (start arm) and allowed to explore the maze with one of the arms closed for 2 min (training trial) for two times, with 1 min of inter-trial interval. Then, the rat is allowed to freely explore all three arms of the maze for 2 min (test trial). The number of entries into and the time spent in each arm is registered and the discrimination ratio [(Time spent in the novel arm—Time spent in the familiar arm)/Total time passed in the two arms] is calculated.

Anymaze video tracking system and software is used to track movement of the rat in the open field test (anxiety/activity test; primary activity outputs—number of steps, distance travelled); in the y-maze (short term/spatial memory test; percentage of time in novel arm); and for NOR and NOL tests.

### Samples to be collected

Brain tissue will be collected. Some rats will be perfused with fixative and the brains kept for possible future immunohistochemistry studies of neuroinflammation. Brain areas from other rats will be frozen for possible future studies on neuroinflammation and alterations in neurotransmission.

Blood will be also taken. Plasma/serum will be collected for analysis of the levels of allopregnanolone (LC-MS), transaminases, bilirubin and inflammatory markers (by ELISA and Western blot).

Liver tissue will be also collected for analysis of liver damage by histology.

### EXAMPLE 4

### Clinical Study

A randomised, double-blind, placebo-controlled, two-part phase 1b/phase 2 study of golexanolone versus placebo among subjects with a history of non-cirrhotic or Child-Pugh class A cirrhotic primary biliary cholangitis (PBC) with clinically significant fatigue and cognitive symptoms on stable background standard of care (SoC) PBC medication will be performed. Pharmacokinetics (PK), safety and tolerability, and preliminary efficacy of golexanolone in this patient population will be evaluated.

The study consists of two separate parts:
PART A: 5 days twice daily (BID) oral administration of golexanolone 40 mg/placebo for assessment of safety and PK.
PART B: 28 days BID oral administration with two dose levels of golexanolone/placebo for safety and preliminary efficacy.

### Investigational Medicinal Product(s), Dosage and Mode of Administration

The Investigational Medical Product (clinical formulation of golexanolone) is supplied as a lipid semi-solid filled in a gelatine capsule for oral administration. For the present study, each capsule contains 10 mg golexanolone. The excipient is a mixture of mono -and diglycerides of capric/caprylic acid (Imwitor® 742), which is of pharmacopoeia quality.

Placebo capsules contain the lipid excipient and are of identical appearance as the golexanolone capsules. The number of placebo capsules administered are adjusted to match the number of golexanolone capsules administered on each dose level. The clinical formulation is described in the table below:

| | | Amount Capric/ | |
|---|---|---|---|
| | | caprylic glycerides | Clinical capsule |
| | Amount | (Imwitor ® 742) | dose |
| Golexanolone | 10 mg | 490 mg | 10 mg capsule |
| Placebo | — | 500 mg | — |

The manufacture of the Investigational Medical Product formulation of golexanolone as used in the present clinical study, may be prepared as described in Example 10 of Applicant's published application WO2019102040.

### Study Design

Subjects are screened for eligibility according to study-specific inclusion/exclusion criteria prior to the first administration of the Investigational Medicinal Product (Visit 1; Screening visit).

Part A: Eight evaluable subjects will be required. Assuming a screen failure rate of 50%, it is estimated that approximately 16 subjects will be screened. Treated subjects withdrawn for other reasons than AE (adverse events) might be replaced, if recommended by the internal safety review committee (iSRC).

Part B: Approximately 84 evaluable subjects will be required. Assuming a 10% drop-out rate, up to 93 subjects will be randomised. Assuming a screen failure rate of 50%, it is estimated that approximately 186 subjects will be screened.

When approximately 60 subjects from Part B completed Day 28 (i.e. 20 subjects per treatment arm), an interim analysis will be performed for sample size re-assessment. A maximum of 126 evaluable subjects may be included, dependent upon results of the re-estimation. Assuming a 10% drop-out rate, up to 139 subjects may be randomised.

Both study parts (A and B) will be placebo-controlled and randomised. In order to minimise potential bias or placebo effect, all study personal, including Sponsor, CRO and Investigators will be blinded.

Subjects participating in Parts A+B will receive IMP for a period of 33 days (Days 1-5 in Part A and Days 1-28 in Part B). Subjects participating in Part A only will receive IMP for a total of 5 consecutive days. Subjects participating in Part B only will receive IMP for a total of 28 consecutive days. To facilitate visit planning, a deviation of +2 days for Day 28 will be acceptable.

### Study Part A

Eight eligible male and female subjects will be randomised on Day 1 (Visit 2) to 5 days of daily administration of either 40 mg BID golexanolone (6 subjects) or placebo BID (2 subjects). PK sampling will occur frequently during the 12 hours following the first dose on Day 1, daily prior to the morning dose on Days 2 through 5, and frequently during the 48 hours following the second dose on Day 5 (evening dose). Subjects will be domiciled at the study site Days 1-7. Safety parameters will be monitored at pre-specified time-points.

A follow-up visit (Visit 3) will be performed on Day 21 (+3 days), i.e. approximately 2 weeks after last

IMP (Investigational Medical Product) administration.

### Primary objective Part A

To assess the safety and tolerability of treatment with 40 mg golexanolone BID for 5 days in non-cirrhotic or Child-Pugh class A cirrhotic PBC subjects with clinically significant fatigue and cognitive symptoms.

### Study Part B

Assuming that safety and PK data from Part A are judged satisfactory, Part B will commence and approximately 93 subjects, assuming a 10% drop-out rate, will be randomised (1:1:1) (i.e. the same number of patients will be randomized for placebo: dose group 1: dose group 2) on Day 1 (Visit 2) to administration of the anticipated dose levels of 40 mg or 80 mg golexanolone BID or placebo BID for 28 consecutive days.

The dosing will be as follows:
1. 40 mg golexanolone BID (i.e. 80 mg per day) in combination with Standard of Care therapy; or
2. 80 mg golexanolone BID (i.e. 160 mg per day) in combination with Standard of Care therapy; or
3. placebo BID in combination with Standard of Care therapy.

Standard of Care therapy may be any one of an oral antioxidant, fluvoxamine, fluoxetine, ondansetron, colchicine, UDCA (ursodeoxycholic acid), OCA (obeticholic acid), ciclosporin, nalmefene, modafinil, rituximab, lactulose, rifaximin, propranolol, furosemide, methotrexate; or any combination thereof.

Adjustments of these pre-defined dose levels might be needed based on the Part A exposure. However, the maximum daily dose is not to exceed 80 mg BID. There will be an interval of at least 1 month between last dose in Part A and first dose in Part B.

The IMP will be self-administered by the subject at home during Part B, except for the study days of visiting the clinic (Days 1, 7, 14, and 28), when the IMP (morning dose) will be administered under supervision. A follow-up safety phone call will be performed on Day 21.

Intake of study medication, changes in concomitant medications and medical events occurring between visits will be registered daily by the subject in an electronic diary. A follow-up visit will be performed on Day 42 (±3 days), i.e. approximately 2 weeks after last IMP administration.

When approximately 60 subjects from Part B completed Day 28 (i.e. 20 subjects per treatment arm) an interim analysis will be performed for sample size re-assessment based on the effects of study drug on PBC-40 cognitive domain.

Blood samples are drawn for serum levels of allopregnanolone.

### Endpoints

1. Frequency, intensity, and seriousness of AEs (Adverse Events), changes from baseline to Day 28 in laboratory parameters and clinical safety parameters.

2. Change from baseline to Day 28 in the following HRQOL (Health-Related Quality of Life) measures:
i. PBC-40 scores for each of the domains (cognition, itch, fatigue, social, emotional, and general symptoms).
ii. EQ-5D-3L tool (described below).

3. Change from baseline to Day 28 in daytime sleepiness related symptoms using the Epworth Sleepiness Scale (ESS), described below.

4. Change from baseline to Day 28 in a battery of cognitive tests, including:
1. Portosystemic Hepatic Encephalopathy Score (PHES) total score (described below).
II. Rey Auditory Verbal Learning test (RAVLT) (described below).
III. Delis and Kaplan Executive Function System (D-KEFS) Letter and Category fluency subtests (described below).

5. Clinical Global Impression of change, PBC version (CGI-C-PBC) (described below).

6. Lowest plasma concentration before the next dose (C_{trough}) will be assessed pre-dose on Days 1, 7, 14 and 28.

### Exploratory Endpoints

1. Plasma concentrations of allopregnanolone

2. Change from baseline to Day 28 in plasma concentrations of pro-inflammatory biomarkers

### Clinical Efficacy Assessments

Objectives

1. To assess effects of golexanolone on health-related quality of life (HRQOL), including fatigue.

2. To assess effects of golexanolone on day-time sleepiness.

3. To assess effects of golexanolone on cognitive function.

4. To evaluate the Investigator's overall impression of treatment effect.

5. To assess the exposure of two dose levels of golexanolone in the target population treated for 28 days.

### Endpoints

1. Change from baseline to Day 28 in the following HRQOL measures:
PBC-40 scores for each of the domains (cognition, itch, fatigue, social, emotional, and general symptoms).
EQ-5D-3L tool.

2. Change from baseline to Day 28 in daytime sleepiness related symptoms using the Epworth Sleepiness Scale (ESS).

3. Change from baseline to Day 28 in a battery of cognitive tests, including:
Portosystemic Hepatic Encephalopathy Score (PHES) total score.
Rey Auditory Verbal Learning test (RAVLT).
Delis and Kaplan Executive Function System (D-KEFS) Letter and Category fluency subtests.

4. Clinical Global Impression of change, PBC version (CGI-C-PBC).

5. Lowest plasma concentration before the next dose (Ctrough) will be assessed pre-dose on Days 1, 7, 14 and 28.

### Exploratory objective

To assess the plasma levels of allopregnanolone and pro-inflammatory biomarkers.

### PBC-40

The PBC-40 is a patient-derived, disease specific HRQOL measure developed and validated for use in PBC. The PBC-40 questionnaire is used in assessing fatigue and cognitive symptoms in PBC, using the disease specific PBC-40 scale with specific domains for cognitive function and fatigue respectively. The PBC-40 questionnaire may also include itching, social, emotional and general PBC related symptoms.

The questionnaire consists of six so called domains of which cognitive function and fatigue are two such domains (the four other domains related to emotional, social, itch and general symptoms). The PBC-40 questionnaire is validated and commonly used and understood by skilled persons within the medical field of PBC (Jacoby et al; Gut 2005;54: pp. 1622-1629). The characteristics of the measure have been described in detail and it has been widely applied in practice (see e.g. Newton J L et al; J Hepatol 2006; 44: pp. 776-782).

It is designed for self-completion by patients and takes approximately 5 minutes to complete.

The paper questionnaire consists of 40 questions, each scored on a scale of 1 to 5 (where 1=least impact, 5=greatest impact) grouped into six domains (cognition, itch, fatigue, social, emotional, and general symptoms). For each domain, scoring involves summing individual question response scores, range of 6-30. Higher scores indicate a poorer quality of life.

The original version of the PBC-40 with a 4-week recall period will be used at screening. An adaptation of the PBC-40 to a weekly recall period will be used at subsequent study visits.

The subject is given the following instruction:
For each statement, please indicate the response that comes closest to how you feel. If any of the statements do not apply to you, please indicate “does not apply”.

### The EQ-5D-3L Tool

The EQ-5D tool is a standardised measure of health status developed by the EuroQol Group to provide a simple, generic measure of health for clinical and economic appraisal (Rabin R et al; Ann Med. 2001 Jul;33(5): pp. 337-343).

The EQ-5D three-level version (EQ-5D-3L) essentially consists of two pages: the EQ-5D descriptive system and the EQ-5D visual analogue scale (EQ VAS):
The EQ-5D-3L descriptive system comprises the following five dimensions, each describing a different aspect of health: MOBILITY, SELF-CARE, USUAL ACTIVITIES, PAIN/DISCOMFORT and ANXIETY/DEPRESSION. Each dimension has three levels: no problems, some problems, extreme problems (labelled 1-3). The respondent is asked to indicate his/her health state by checking the box against the most appropriate statement in each of the five dimensions.
The EQ VAS records the respondent's self-rated health on a vertical VAS where the endpoints are labelled The best health you can imagine and The worst health you can imagine. This information can be used as a quantitative measure of health outcome as judged by the individual respondents.

For both the descriptive part of the questionnaire and the VAS, the subject is asked to rate the health of TODAY.

An EQ-5D summary index is derived by applying a formula that essentially attaches values (weights) to each of the levels in each dimension. A paper version will be used.

### Epworth Sleepiness Scale (ESS)

The ESS is a validated self-administered questionnaire for assessment of daytime hypersomnolence (Johns M.; Sleep 1994;17: pp.703-710). Respondents are asked to rate, on a 4-point scale (0-3), their usual chances of dozing off or falling asleep while engaged in eight different activities (i.e. eight questions). The ESS score (the sum of eight item scores, 0-3) can range from 0 to 24. The higher the ESS score, the higher that person's average sleep propensity in daily life, or their ‘daytime sleepiness’.

A paper version of the questionnaire will be used. The questionnaire takes no more than 2-3 minutes to answer.

Interpretation of the scores:

| | |
|---|---|
| 0-5 | Lower Normal Daytime Sleepiness |
| 6-10 | Higher Normal Daytime Sleepiness |
| 11-12 | Mild Excessive Daytime Sleepiness |
| 13-15 | Moderate Excessive Daytime Sleepiness |
| 16-24 | Severe Excessive Daytime Sleepiness |

### Portosystemic Hepatic Encephalopathy Score (PHES)

The PHES is a paper-pencil test battery for the assessment of covert hepatic encephalopathy (CHE) (Weissenborn K., Diagnosis of encephalopathy. Digestion. 1998 Jul;59 Suppl 2:pp.22-24; Weissenborn K, et al; J Hepatol. 2001; May: 34(5):pp. 768-773; Weissenborn K. PHES: J Hepatol. 2008 Sep;49(3): pp.308-312).

It is the only cognitive test battery that has been validated for hepatic indications. However, since cognitive test performance is typically more impaired in covert HE than in PBC, for this study the PHES will be supplemented with additional measures of memory and verbal fluency (see the tests below for Rey Auditory Verbal Learning test and Delis and Kaplan Executive Function System Letter and Category fluency subtests).

The PHES is composed of five sub-tests, number connection test-A (NCT-A), number connection test-B (NCT-B), serial dotting test (SDT), line tracing test (LTT) and digit symbol test (DST). PHES assesses motor speed, motor accuracy, concentration, attention, visual perception, visual-spatial orientation, visual construction, and memory, which are related to most of neuropsychological impairments in CHE.

The subject is given a short, standardised instruction to each of the five subtests and completes them one at a time under supervision. To make sure the subject has understood correctly, training tests are completed for the NCT-A, the SDT, LTT and DST tests.

Scoring of the PHES: The time (seconds) spent on each test is noted and converted into a score as indicated in the age-adjusted normal values. A score of zero is given for a subtest performance within +/−1 standard deviation (SD). The maximum score is +1 while the lowest possible score is −3 (−3 SDs). Note that two scores are assigned for the LTT: A time score and an error score. The sum of the 6 sub-scores is the PHES and is abnormal when below −4. The PHES range is −18-6. Testing and scoring takes approximately 15 minutes.

### Rey Auditory Verbal Learning test (RAVLT)

The RAVLT is a validated word list learning task composed of 15 words that are read to the subject at a rate of one word/second after which the subject is asked to recall as many words as they can. This procedure is repeated five times. The sum of words recalled across these five trials constitutes the immediate recall score. This is a common testing paradigm for the measurement of verbal episodic memory. The RAVLT is a widely used test, originally adapted from the French in 1959 (Schmidt et al. Rey Auditory Verbal Learning Test: A Handbook (published by Western Psychological Services).

The RAVLT will be given before the PHES (as a measure of learning). After completion of the PHES, the RAVLT delayed recall test will be given in which the subject is asked to recall as many words from the list as possible after the delay.

There are several equivalent forms permitting alternation between forms for repeated administration. The form used at visit 2 (Baseline) should be same as form used at Visit 6 (end of treatment).

Administration time for both immediate and delayed forms is 8-10 minutes.

### Delis and Kaplan Executive Function System Letter (D-KEFS) and Category Fluency Subtests

The Category Fluency and Letter Fluency tests from the D-KEFS are validated ideational fluency tasks in which the subject is asked to say as many words as possible in 1 minute that adhere to a particular rule.

For the Category Fluency task, first the subject is asked to name as many animals as they can in 1 minute. Then a second category is provided (boy's names). The score is the sum of the number of words provided in the two categories. An alternative version using two different and equivalent categories will be given at alternating visits.

Letter Fluency requires the subject to say as many words as possible beginning with a target letter in 1 minute. The number of words generated in three trials with three different letters are summed.

Constraints are given (e.g. no proper names, no numbers) and only words within those rules are counted as correct.

Together these tests take 6-7 minutes to administer and another 3 minutes to score. For both the Letter and Category fluency tasks there are two equivalent forms that can be alternated. The form used at Visit 2 (Baseline) should be same as form used at Visit 6 (end of treatment). The alternate form can be used at Screening and Visit 4.

### Clinical Global Impression of Severity and Change-PBC Version

The clinical global impression (CGI) scale consists of two clinician-rated instruments, CGI-S-PBC and CGI-C-PBC, respectively measuring overall disease severity and change. A PBC-specific version has been developed based upon extensive qualitative work in PBC of Jacoby 2005 (Gut 2005;54:pp. 1622-1629) that underpins the well validated PBC-40 and employing these domains and questions in a semi-structured clinical interview format with anchored clinician ratings. This PBC specific version of the CGI-S and CGI-C is undergoing validation, and is described in detail below.

Since its first publication the CGI has become one of the most widely used assessment tools in psychiatry. For example, the CGI, especially the CGI change scale (CGI-C) has been widely utilised as an efficacy measure in clinical drug trials in different mental disorders (e.g. depression, schizophrenia). Its popularity is mainly based on its conciseness and its ability to capture a well-informed overall clinical impression, even in disease states where the presentation is heterogeneous.

In psychiatric indications, the CGI is administered after completing several standardised semi-structured interview instruments to survey symptoms and their severity, thus assuring that CGI ratings are based upon a uniform examination of the subjects across Raters, sites, and visits. Reliable use of a CGI in the absence of preceding clinical rating scales can be achieved by providing disease-specific structure (Kolevzon A et al; J Neurodev Disord. 2021 Jan 4;13(1):p. 3).

To support the comparison of clinical status at baseline, the CGI-S-PBC involves a semi-structured interview with the subject which will be performed at baseline, and which covers the domains previously identified as clinically meaningful for PBC. Extensive observational notes and subdomain ratings from the CGI-S-PBC will be reviewed by the Rater before the interview for CGI-C-PBC is conducted so that ratings of change are well informed by the degree of severity and symptom profile observed prior to treatment.

### Exploratory Efficacy Assessments

### Plasma Levels of Allopregnanolone and Pro-inflammatory Biomarkers

Blood samples for analysis of plasma levels of allopregnanolone and pro-inflammatory biomarkers will be collected in study Part B. The samples will be registered in a tissue-bank, where required by local legislation, and stored at −70° C. until analysed at the end of the study.

Clinical Global Impression of Change—Primary Biliary Cholangitis (CGI-C-PBC)

The CGI-C-PBC is a clinical rating scale designed by the present patent applicant, to capture the rater's clinical impression of the magnitude of change from the initiation (baseline) of treatment. The CGI-C-PBC rating ranges from 1 (very much improved) to 7 (very much worse), with a midpoint rating of 4 to reflect no change from baseline. The CGI-C-PBC is completed during the follow up visits but not at Baseline. A window of the past 4 weeks is to be used for recall.

To render reliable CGI-C-PBC ratings (C stands for change), a thorough review of baseline CGI-S-PBC (S stands for start) Investigator Notes is required. Hence optimal CGI-C-PBC ratings rely on thorough baseline Investigator notes that describe in detail symptoms and signs, their severity, frequency and functional consequence over the previous 4 weeks in each of the symptom domains.
1. Referring closely to each domain's CGI-S-PBC (described below) Investigator notes as a guide, and focusing on observations during the previous 4 weeks, raters should interview study participants (and if appropriate any additional informants) focusing specifically on capturing change in the severity, frequency and functional consequences of those symptoms and signs that were described in the CGI-S-PBC Investigator notes section.
2. Raters should take into account information from all sources, including their own objective observations of the study participant per domain.
3. All changes from baseline should be described in detail on the CGI-C-PBC Investigator Notes form. The narrative on this form constitutes important justification for the CGI-C-PBC rating which is an important endpoint in this study. Hence a thorough narrative description of the change (precisely what is different now, relative to the descriptions provided at baseline), is required.
4. Once the narrative for each of the domains has been completed, the rater should assign a CGI-C-PBC score for that domain.
5. Finally the rater should provide an overall CGI-C-PBC score. In rendering this rating, the rater should consider the amount, consistency and overall clinical meaningfulness of change from baseline across all domains. Note that the OVERALL CGI-C-PBC score is not an algorithmic computation from the individual domain scores. It is a clinical judgement in the aggregate, i.e. a dramatic change on one domain can be of great clinical significance for a given patient.

### The CGI-C-PBC Rating Guide is as Follows

The clinician will rate each PBC-40 domaine element:
1. Itch (Itch disturbing sleep, scratching until skin is raw, embarrassment).
2. Fatigue (Force self out of bed, nap, fatigue, worn out, force self to do things, go to bed early, sudden onset of fatigue, drained of energy, takes a long time to do things, need a day to recover after a busy day, need to pace self).
3. Cognitive symptoms (Short concentration span, remembering things one day to the next, trouble keep up conversations, difficult to concentrate, remember what you wanted to do?).
4. Psychosocial consequences (Neglecting family, feel guilty, sex life, can't go out, keep PBC to self, unable to plan holidays, can't need a normal life, reduced quality of life).
5. Emotional Distress (More stressed about things than before, PBC gets me down, worry about PBC future).
6. Bodily Symptoms Typical for PBC (Able to eat, bloated when drink, problems with alcohol, physical discomfort, right side, dry mouth, aches in long bones of arms/legs).

1=Very Much Improved
Marked improvement in frequency and severity, very significant functional improvement.
Improvement likely to be noticeable in the clinic as well.

2=Much Improved
Moderate improvement in both frequency and severity.
Improvements have clear functional consequences.

3=Minimally Improved
Mild or modest improvement on either frequency, severity or both.
Degree of Improvement must be clinically meaningful.
Improvement represents more than random chance variation.

4=No Change
Absence of change in symptoms, functioning or clinical presentation.
Slight variation in either direction, of a magnitude that is likely due to chance.
Fluctuations or day-to-day changes.
Changes that are possibly present, but not certain.

5=Minimally Worse
Some worsening on either frequency or severity or both.
Degree of Worsening must be clinically meaningful.
Worsening represents more than random chance.

6=Much worse
Moderate worsening, involving both frequency and severity.
Degree of worsening has clear functional consequences.

7=Very Much Worse
Marked worsening in both frequency and severity, very significant functional worsening.
Worsening likely to be noticeable in the clinic as well.

The clinician will finally sum up a total score based on the rating for each PBC-40 domaine element.

### Clinical Global Impressions-Severity—Primary Biliary Cholangitis (CGI-S-PBC)

A clinical global impression of the severity of a symptom is a key step in assessing the response to treatment of that symptom, and is a necessary step for regulatory approval of a new treatment. This tool, the CGI-S-PBC, has been designed by the present patent applicant, to enable such an assessment in primary biliary cholangitis (PBC).

The completed CGI-S-PBC should reflect for a study participant the clinicians view of the overall degree, severity and functional impact of primary biliary cholangitis (PBC) symptoms over the past month. This is recorded using a single value on a 7-point scale, where 1 indicates normality (typical of the normal population) and 7 indicates the most severe a clinician is likely to ever see among PBC patients in the clinic.

This guidance section provides a semi-structured interview and rating guide to assist the clinician-rater, to systematically review for each participant, how each of the domains that have been judged to be clinically meaningful in PBC are affected. Severity “anchors” within each of these domains are also provided to guide the clinician's rating and to assure consistent calibration of the rating levels between patients, clinicians and sites.

Raters should be clinicians who have sufficient familiarity with PBC patients to judge the relative severity distinctions outlined below. They should also have been trained in the use of this scale.

The following steps are required for reliable use of the scale:
1. For each symptom domain listed on the CGI-S-PBC Investigator Notes form below, raters should interview the patient with the goal of arriving at a clinical description that can support one of the 7 rating levels offered. Suggested Prompting Questions are offered for each domain and should be used as a guide.
2. When assigning a rating for each domain, raters should take into account information from all available sources including principally interview of the study participant.
3. The reference for comparison (i.e. a rating of 1) is a comparable individual (in terms of age and gender) without PBC and in overall good health.
4. A window of 4 weeks before the clinic visit should be used as the recall period.
5. The CGI-S-PBC Investigator Notes section should thoroughly capture sufficient clinical detail what happened and when, what is typical, what is occasional, what happened over the past 4 weeks but is rare, to enable the study team to reconstruct how each domain was manifested. This is critical as it establishes the pre-treatment baseline for subsequent rating of the change score.
6. When raters have finished recording their notes for each domain, they should enter a Domain Severity score based on the descriptions within the anchors. Anchors contain examples of severity characterizations for each domain, however other examples in their respective domains should be included as deemed clinically appropriate and described in the notes section provided.
7. The characterizations within the PBC-specific anchors may not describe any given subject precisely. Ratings should reflect the overall perceived severity within each symptom complex, avoiding disproportionate weighting of minor or rarely occurring symptoms that may be less clinically meaningful.
8. Finally, based upon their clinical judgement, the rater should render a CGI-S-PBC Overall score using all available input. Note that the CGI-S-PBC Symptoms Overall score rating reflects clinical judgement and is not an algorithmic computation (i.e. mean or mode) derived from symptom domain scores). In the end, the CGI-S-PBC Symptoms Overall score should be a clinical impression that reflects the rater's estimate of overall severity at that point in time.

### The CGI-S-PBC Rating guide is as Follows

The clinician will rate each PBC-40 domaine element:
1. Itch (Itch disturbing sleep, scratching until skin is raw, embarrassment).
2. Fatigue (Force self out of bed, nap, fatigue, worn out, force self to do things, go to bed early, sudden onset of fatigue, drained of energy, takes a long time to do things, need a day to recover after a busy day, need to pace self).
3. Cognitive symptoms (Short concentration span, remembering things one day to the next, trouble keep up conversations, difficult to concentrate, remember what you wanted to do?).
4. Psychosocial consequences (Neglecting family, feel guilty, sex life, can't go out, keep PBC to self, unable to plan holidays, can't need a normal life, reduced quality of life).
5. Emotional Distress (More stressed about things than before, PBC gets me down, worry about PBC future).
6. Bodily Symptoms Typical for PBC (Able to eat, bloated when drink, problems with alcohol, physical discomfort, right side, dry mouth, aches in long bones of arms/legs).

After using prompting questions relevant to each PBC symptom below, based upon number, severity and consequence of episodes during the past month, the clinician will rate each PBC symptom.

### 1. Itch Typical of PBC

Suggested Itch Prompting Questions:
How much has itching been a problem?
Has itching disturbed your sleep? How often? How severe? (Do you get at least some sleep?)
Do you scratch so much you make your skin raw?
Does the compulsion to scratch cause embarrassment?

Severity=1: Normal, not at all impaired: Typical.

Severity=2: Borderline, slightly distressing: May interfere with day to day functioning.

Severity=3: Mildly distressing/impaired: Mildly distressing itching. Somewhat or occasionally interferes with day to day functioning.

Severity=4: Moderate distressing/impaired: Moderately distressing severity. Distracting with clear functional consequences (i.e. on several days had to reschedule planned events as a result of itching/scratching self).

Severity=5: Markedly distressing/impaired: Markedly distressing. Discomfort often interferes with day to day functioning to a marked degree (i.e. cancelled plans, unable to work or perform productive activity as a result of itching/scratching self).

Severity=6: Severely impaired: Severe distress on most days itching. Interferes with functioning (i.e. no longer make plans or expect to be productive as a result of itching/scratching self).

Severity=7: Among the most severely impaired: Profound unrelenting itching makes normal day to day functioning impossible. Essentially disabled as a result of itching/scratching self.

### 2. Fatigue

Suggested Fatigue Prompting Questions:
How often (how many days) do you have to force yourself to get out of bed?
Do you have to have a sleep/nap during the day?
How often did fatigue interfere with your daily routine? How? Can you give examples?
How often do you feel worn out? Please describe.
Do you feel so tired, you have to force yourself to do the things you needed to do?
Have you felt so tired, you had to go to bed earlier than usual?
Does fatigue sometimes come on suddenly?
Do you sometimes feel like PBC has drained you of energy? Please give examples from the past month?
Do you feel it takes you a long time to do things? Please give examples. How long? Is it every day?
Does it happen that after a busy day, you need at least another day to recover? How often? Can you give a recent example ?
Do you have to pace yourself to be able to get things done?

Severity=1: Normal, not at all impaired: Normal; typical.

Severity=2: Borderline, slightly distressing: May interfere with day to day functioning.

Severity=3: Mildly distressing/impaired: Mildly severe overall or moderately severe one fatigue symptom or occasionally severe. Mildly interferes with day to day functioning.

Severity=4: Moderate distressing/impaired: Moderately distressing fatigue severity on more than one symptom. Clear functional consequences (i.e. on several days had to reschedule planned events).

Severity=5: Markedly distressing/impaired: Markedly distressing fatigue symptoms reflected in multiple ways) Fatigue often interferes with day to day functioning to a marked degree (i.e. cancelled plans, unable to work or perform productive activity).

Severity=6: Severely impaired: Severe fatigue reflected broadly on most days. Interferes with day to day functioning most days (i.e. no longer make plans or expect to be productive as a result).

Severity=7: Among the most severely impaired: Profound fatigue, fatiguability, lack of energy making most normal day to day functioning impossible. Essentially disabled as a result of fatiguability and its consequences.

### 3. Cognitive Symptoms

Suggested Cognitive Symptoms Prompting Questions:
Have you felt your concentration span was short because of PBC. Please describe with examples. Is it every day?
Have you had difficulty remembering things from one day to the next? Do you fail to remember or with some effort you can manage? How much effort?
Have you found it can be difficult to keep up with conversations? Is this because of problems with conversations? Can you please describe?
Do you find it difficult to concentrate?
Do you find often it is difficult to remember what you wanted to do?

Severity=1: Normal, not at all impaired: Normal; typical.

Severity=2: Borderline, slightly distressing: May interfere with day to day functioning. Functioning is sometimes effortful.

Severity=3: Mildly distressing/impaired: Memory and/or concentration problems are mild most of the time, or moderately severe intermittently. Functioning is often effortful. Interferes with day to day functioning about once a week.

Severity=4: Moderate distressing/impaired: Moderately severe memory and/or concentration problems. Clear functional consequences (i.e. on several days had to reschedule planned events).

Severity=5: Markedly distressing/impaired: Markedly severe memory and/or concentration problems. Often interferes with day to day functioning to a marked degree (i.e. cancelled plans, many days unable to work or perform productive activity).

Severity=6: Severely impaired: Extremely severe memory and/or concentration problems felt on most days. Interferes with day to day functioning most days. (i.e. no longer makes plans or expects to be productive as a result).

Severity=7: Among the most severely impaired: Profound subjective cognitive dysfunction affecting concentration and memory, making most normal day to day functioning impossible. Essentially disabled as a result of cognitive dysfunction and its consequences.

### 4. Psychosocial Consequences

Suggested Psychosocial consequences Prompting Questions:
Have you felt you neglect your family because of having PBC? What are some recent examples?
Do you sometimes feel guilty that you can't do things you used to do because of having PBC? What are some things in the past month you could not do that made you feel guilty?
Has your sex life been negatively affected by having PBC during the past month?
During the past month have you felt frustrated that you can't just go out and enjoy yourself because you have PBC? Can you give an example or two?
Do you tend to keep the fact that you have PBC to yourself? If so, why?
Do you feel that having PBC has made you unable to plan holidays? How has it affected your social life over the past month? Are there any parts of your life that, over the past month, remained unaffected by PBC?
Can you still lead a normal life? Has PBC reduced your quality of life? How? Can you give some examples from the past month?

Severity=1: Normal, not at all impaired: Normal; typical.

Severity=2: Borderline, slightly distressing: May interfere with day to day functioning.

Severity=3: Mildly distressing/impaired: Mildly severe overall or moderately severe intermittently on psychological or social consequences of living with PBC, or very occasionally severe.

Severity=4: Moderate distressing/impaired: Moderately severe psychosocial consequences. Distress or isolation occasionally results in functional consequences (i.e. on several days this month, unable to be normally socially interactive).

Severity=5: Markedly distressing/impaired: Markedly severe psychosocial consequences. Distress or isolation often interferes with day to day functioning to a marked degree (i.e. cancelled plans, often unable to be normally socially interactive).

Severity=6: Severely impaired: Severe psychosocial consequences felt on most days. Level of distress or isolation interferes with day to day functioning most days. (i.e. no longer make plans or expects to normally socially interact as a result).

Severity=7: Among the most severely impaired: Profound psychosocial consequences causing distress and/or isolation most days, normal social relationships impossible. Essentially disabled as a result of severe psychosocial distress and/or isolation.

### 5. Emotional Distress

Suggested Emotional Distress Prompting Questions:
Would you say that because of PBC you get more stressed about things than you used to? Can you give some examples from the past month?
Does having PBC get you down? Can you please describe this to me?
Do you worry about how your PBC will be in the future? How it will affect you? How often do you worry about this?

Severity=1: Normal, not at all impaired: Normal; typical.

Severity=2: Borderline, slightly distressing: Occasional distress. May interfere with day to day functioning.

Severity=3: Mildly distressing/impaired: Mild consistent emotional distress or moderately severe intermittent emotional distress.

Severity=4: Moderate distressing/impaired: Moderately severe emotional distress. Distress itself occasionally results in functional consequences (i.e. on several days emotional distress impairs functioning).

Severity=5: Markedly distressing/impaired: Markedly severe emotional distress felt several times a week, often resulting in functional consequences. (i.e. cancelled plans, often unable to be normally socially interactive).

Severity=6: Severely impaired: Severe emotional distress felt on most days. Level of sadness, emotional stress, worry about the future interferes with day to day functioning most days.

Severity=7: Among the most severely impaired: Profound emotional distress, virtually continuous. Level of sadness, emotional stress, worry about the future responsible for almost complete disability.

### 6. Body Symptoms Typical of PBC

Suggested Somatic Symptoms Prompting Questions:
To what degree have you been able to eat what you like?
Do you feel bloated when you drink only a small amount? Please describe? (How much drink, how bloated, how often?)
How does alcohol make you feel? Have you had any alcohol in the past month? Why/why not? If yes, how did it make you feel?
Have you had any physical discomfort? (more on the right side?) Please describe severity and frequency.
Have you had dry eyes? (If yes, please describe. Include severity and frequency).
Has your mouth been dry? (If yes, please describe. Include severity and frequency).
Have you had aches in the long bones of your arms and legs? (If yes, please describe. Include severity and frequency).

Severity=1: Normal, not at all impaired: Normal; typical.

Severity=2: Borderline, slightly distressing: May interfere with day to day functioning.

Severity=3: Mildly distressing/impaired: Mildly distressing symptoms or moderately distressing on one symptom. Mildly interferes with day to day functioning.

Severity=4: Moderate distressing/impaired: Moderately distressing severity on more than one symptom. Clear functional consequences (i.e. on several days had to reschedule planned events).

Severity=5: Markedly distressing/impaired: Marked distress on more than one symptom. Pain or discomfort often interfere with day to day functioning to a marked degree (i.e. cancelled plans, unable to work or perform productive activity).

Severity=6: Severely impaired: Severe distress on more than one symptom most days. Interferes with day to day functioning most days. Functioning is markedly affected on most days (i.e. no longer make plans or expects to be productive as a result).

Severity=7: Among the most severely impaired: Profound pain, discomfort or somatic symptoms make normal day to day functioning impossible. Essentially disabled as a result of somatic symptoms.

The clinician will finally sum up a total score based on the rating for each PBC-40 domaine element.

### Inclusion Criteria

1. Established diagnosis of PBC based on the presence of 2 out of the 3 key disease characteristics:
1. AMA (anti-mitochondrial antibodies) or PBC-specific ANA (antinuclear antibodies) at a titre of 1/40 or greater;
2. Elevated alkaline phosphatase (above the upper limit of normal (uln) for the relevant laboratory);
3. Compatible or diagnostic liver biopsy;

2. Clinically significant fatigue (defined for the purposes of this study as a PBC-40 Fatigue Domain score of ≥28 at screening) in the setting of clinically significant cognitive symptoms (PBC-40 Cognitive Domain ≥15);

3. Stable UDCA dose for 3 months (either at 13-15mg/Kg) or not on UDCA if intolerant;

4. For females of childbearing potential: Willing to use highly effective contraception or to practice sexual abstinence to avoid pregnancy for entire duration of the treatment period;

5. Age ≥18 and ≤75 (both male and female).

### Exclusion Criteria

1. Liver cirrhosis Childs Pugh B or C disease;

2. Clinical evidence of decompensation (hepatic encephalopathy or variceal bleeding);

3. History of hepatocellular carcinoma;

4. Bilirubin >1.5×ULN;

5. Evidence of biliary obstruction;

6. Concomitant disease characterised by chronic fatigue; 7. Regular use of prescribed or over the counter medications known to cause fatigue or cognitive dysfunction (including, but not limited to, benzodiazepines, opiates other than codeine phosphate, sleeping pills, regular (daily) anti-histamine use (regular daily anti-histamine use in the last four weeks), anti-psychotic agents or recreational drug use);

8. Anticipated change in PBC medication within the duration of the trial;

9. Regular (more than one week per month) alcohol consumption in excess of recommended safe limits (14 units per week);

10. Active participation in another interventional trial or exposure to another experimental drug within 5 half-lives;

11. Pregnancy or planning to get pregnant;

12. Clinical diagnosis of AIH overlap;

13. Concurrent liver disease of another aetiology;

14. Concurrent illness which in the opinion of the investigator is likely to compromise patient safety and/or interpretation of study results.

1. A method for the treatment of fatigue or cognitive impairment, or a combination thereof, in a non-cirrhotic primary biliary cholangitis (PBC) patient, whereby the compound golexanolone (3α-ethynyl-3β-hydroxyandrostan-17-one oxime) of formula (I) 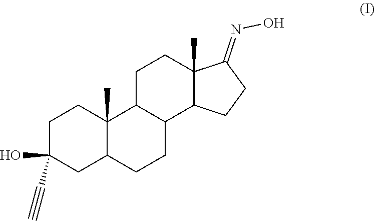 [SMILES: C#C[C@]1(O)CC[C@@]2(C)C(CCC3C2CC[C@]2(C)/C(=N/O)CCC32)C1]
or a pharmaceutically acceptable salt thereof, is administered to a patient in need of such treatment.

2. (canceled)

3. The method according to claim 1, wherein the compound golexanolone affects the motor function of the primary biliary cholangitis (PBC) patient.

4. The method according to claim 1, wherein the PBC patient further suffers from itching.

5. The method according to claim 1, wherein the PBC patient further suffers from social problems.

6. The method according to claim 1, wherein the PBC patient further suffers from emotional problems.

7. The method according to claim 1, wherein the patient further suffers from excessive daytime sleepiness (EDS).

8. The method according to claim 1, wherein the patient has a PBC-40 Cognitive Domaine which is 15 or higher (moderate); or in the range 15-21; or 21 or higher (severe).

9. The method according to claim 1, wherein the patient has a PBC-40 Fatigue Domaine which is 28 or higher (moderate); or in the range of 28-39; or 39 or higher (severe).

10.-13. (canceled)

14. The method according to claim 1, wherein said treatment is combination treatment with at least one more compound useful in the treatment of primary biliary cholangitis (PBC).

15. The emethod according to claim 14, wherein the at least one more compound is selected from any one of an oral antioxidant, fluvoxamine, fluoxetine, ondansetron, colhicine, UDCA (ursodeoxycholic acid), OCA (obeticholic acid), ciclosporin, nalmefene, modafinil, rituximab, lactulose, rifaximin, propranolol, furosemide, methotrexate, or a fibrate (such as bezafibrate and fenofibrate); or any combination thereof.

16.-17. (canceled)

18. The method according to claim 1, wherein said patient has a serum level of allopregnanolone which is 0.03 or higher.

19. The method according to claim 1, wherein the patient further suffers from motor impairment.

20. The method according to claim 19, wherin the PBC patient further suffers from itching, social problems, emotional problems, or excessive daytime sleepiness (EDS).

21. The method accoriding to claim 19, wherein the patient has a PBC-40 Cognitive Domaine which is 15 or higher (moderate); or in the range 15-21; or 21 or higher (severe).

22. The method according to claim 19, wherein the patient has a PBC-40 Fatigue Domaine which is 28 or higher (moderate); or in the range of 28-39; or 39 or higher (severe).

23. The method accoriding to claim 19, wherein said treatment is a combination treatment with at least one more compound useful in the treatment of primary biliary cholangitis (PBC).

24. The method according to claim 23, wherein the at least one more compound is selected from any one of an oral antioxidant, fluvoxamine, fluoxetine, ondansetron, colhicine, UDCA (ursodeoxycholic acid), OCA (obeticholic acid), ciclosporin, nalmefene, modafinil, rituximab, lactulose, rifaximin, propranolol, furosemide, methotrexate, or a fibrate (such as bezafibrate and fenofibrate); or any combination thereof.

25. The method according to claim 19, wherein said patient has a serum level of allopregnanolone which is 0.03 or higher.

26. The method according to claim 20, wherein the patient has a PBC-40 Cognitive Domaine which is 15 or higher (moderate); or in the range 15-21; or 21 or higher (severe).

27. The method according to claim 20, wherein said patient has a serum level of allopregnanolone which is 0.03 or higher.
